(19) United States
(12) Patent Application Publication
(10) Pub. No.: US 2022/0240822 A1
(43) Pub. Date: Aug. 4, 2022

(54) DETECTING DEVICE AND MEASURING DEVICE

(71) Applicant: SEIKO EPSON CORPORATION, Tokyo (JP)

(72) Inventors: Hiromu TAKAYAMA, CHINO-SHI (JP); Takefumi FUKAGAWA, NAGANO-KEN (JP)

(73) Assignee: SEIKO EPSON CORPORATION, Tokyo (JP)

(21) Appl. No.: 17/586,755

(22) Filed: Jan. 27, 2022

(30) Foreign Application Priority Data
Jan. 29, 2021 (JP) 2021-013630

(51) Int. Cl. A61B 5/1455

(52) U.S. Cl. CPC A61B 5/14552; A61B 2562/0242; A61B 2562/0238

(57) ABSTRACT

A detecting device includes: a first light-emitting unit configured to emit first light having a green wavelength band; a second light-emitting unit configured to emit second light having a wavelength band higher than that of the green wavelength band; and a light-receiving unit configured to receive the first light emitted from the first light-emitting unit and emitted from a living body and the second light emitted from the second light-emitting unit and emitted from the living body. The light-receiving unit includes a first light-receiving region configured to receive the first light, a second light-receiving region provided at a position farther away from the first light-emitting unit than the first light-receiving region and configured to receive the second light, and a first filter provided in one of the first light-receiving region and the second light-receiving region and configured to selectively transmit light in a corresponding wavelength band.

The present application is based on, and claims priority from JP Application Serial Number 2021-013630, filed Jan. 29, 2021, the disclosure of which is hereby incorporated by reference herein in its entirety.

### BACKGROUND

### 1. Technical Field

The present disclosure relates to a detecting device and a measuring device.

### 2. Related Art

Various measurement technologies for non-invasively measuring biological information such as pulse waves have been proposed in the past. For example, JP-A-2018-061675 discloses a detecting device that includes a light-emitting unit configured to eject light to a living body, and a light-receiving unit configured to receive light ejected from the light-emitting unit and reflected by the living body to be incident on the light-receiving unit. JP-A-2018-061675 discloses a technology whereby the light utilization efficiency of the light-emitting unit is increased and a measure against stray light for the light-receiving unit is implemented by installing a light-shielding member between the light-emitting unit and the light-receiving unit in the detecting device.

However, for the detecting device described above, there is a problem in that the device configuration cannot be downsized because a plurality of light-receiving units that receive light reflected by a living body need to be provided.

### SUMMARY

According to one aspect of the present disclosure, there is provided a detecting device including: a first light-emitting unit configured to emit first light having a green wavelength band, a second light-emitting unit configured to emit second light having a wavelength band higher than that of the green wavelength band, and a light-receiving unit configured to receive the first light emitted from the first light-emitting unit and emitted from a living body and the second light emitted from the second light-emitting unit and emitted the living body, wherein the light-receiving unit includes a first light-receiving region configured to receive the first light, a second light-receiving region provided at a position farther away from the first light-emitting unit than the first light-receiving region and configured to receive the second light, and a first filter provided in one of the first light-receiving region and the second light-receiving region and configured to selectively transmit light in a corresponding wavelength band.

According to one aspect of the present disclosure, there is provided a measuring device including: a detecting device according to the above-described aspect, and an information analysis unit configured to identify biological information from a detection signal indicating a detection result by the detecting device.

### BRIEF DESCRIPTION OF THE DRAWINGS

FIG. 1 is a side view of a measuring device according to a first embodiment.

FIG. 2 is a configuration diagram focused on a function of a measuring device.

FIG. 3 is a plan view of a detecting device.

FIG. 4 is a cross-sectional view taken along the line IV-IV in FIG. 3.

FIG. 5 is a graph showing a transmission spectrum of a skin.

FIG. 6 is a graph illustrating a relationship between a red light-emitting unit and a light-receiving unit.

FIG. 7 is a view for explaining an operation of a detecting device.

FIG. 8 is a cross-sectional view of a detecting device according to a second embodiment.

FIG. 9 is a cross-sectional view of a detecting device according to a third embodiment.

FIG. 10 is a cross-sectional view of a detecting device according to a modified example of the third embodiment.

### DESCRIPTION OF EXEMPLARY EMBODIMENTS

Hereinafter, one embodiment of the present disclosure will be described with reference to the accompanying drawings. Note that in each of the figures below, to illustrate each member at a recognizable size, the scale or angle of each member is changed from the actual scale or angle.

### First Embodiment

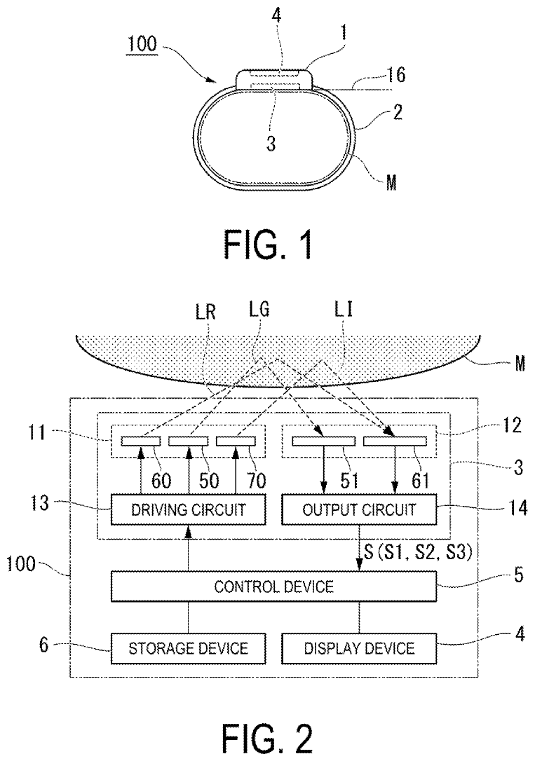

FIG. 1 is a side view of a measuring device 100 according to a first embodiment. The measuring device 100 according to the present embodiment illustrated in FIG. 1 is a biometric instrument that non-invasively measures biological information of a test subject (e.g., a human), which is an example of a living body. The measuring device 100 is mounted at a site (hereinafter referred to as the “measurement site”) M that serves as a measurement target of the body of a test subject. The measuring device 100 according to the present embodiment is a wristwatch-type portable instrument including a housing unit 1 and a belt 2. The measuring device 100 is mountable to a wrist of the test subject by winding a band-shaped belt 2 around a wrist, which is an example of a measurement site (living body) M. In the present embodiment, pulse waves (e.g., pulse peak interval or PPI) and oxygen saturation (SpO2) of the test subject are used as examples of biological information. A pulse wave means temporal change in blood vessel volume linked to the beats of the heart. Oxygen saturation means a percentage (%) of hemoglobin bound to oxygen in total hemoglobin in the blood of the test subject. Oxygen saturation is an indicator for assessing the respiratory function of the test subject.

FIG. 2 is a configuration diagram focused on a function of the measuring device 100. As illustrated in FIG. 2, the measuring device 100 according to the present embodiment includes a control device 5, a storage device 6, a display device 4, and a detecting device 3. The control device 5 and the storage device 6 are installed inside the housing unit 1. As illustrated in FIG. 1, the display device 4 is installed on a surface of the housing unit 1 on a side opposite to the measurement site M. The display device 4 displays various images including measurement results under the control of the control device 5. The display device 4 is, for example, a liquid crystal display panel.

The detecting device 3 is an optical sensor module that generates a detection signal S in accordance with the state of the measurement site M. As illustrated in FIG. 1, the detecting device 3 is installed, for example, on a surface (hereinafter referred to as the detection surface) 16 of the housing unit 1 that faces the measurement site M. The detection surface 16 is a surface that comes into contact with the measurement site M. As illustrated in FIG. 2, the detecting device 3 according to the present embodiment includes a light-emitting unit section 11, a light-receiving unit 12, a driving circuit 13, and an output circuit 14. Note that one or both of the driving circuit 13 and the output circuit 14 may be installed as an external circuit of the detecting device 3. That is, the driving circuit 13 and the output circuit 14 can be omitted from the detecting device 3.

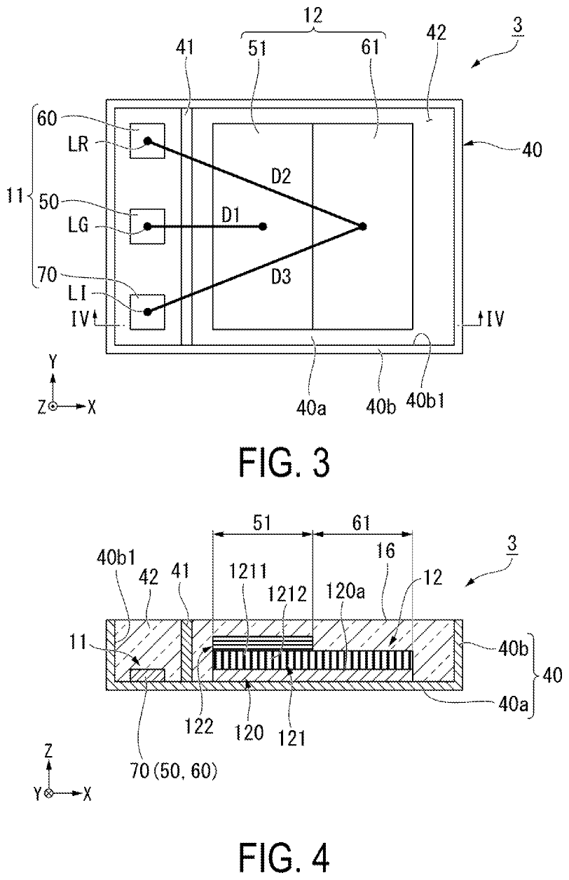

FIG. 3 is a plan view of the detecting device 3. FIG. 4 is a cross-sectional view taken along the line IV-IV in FIG. 3. As illustrated in FIG. 3 and FIG. 4, the detecting device 3 according to the present embodiment further includes, in addition to the light-emitting unit section 11 and the light-receiving unit 12, a case 40, a light-shielding wall 41, and a sealing layer 42. Note that in FIG. 3 and FIG. 4, illustrations of the driving circuit 13 and the output circuit 14 are omitted.

Hereinafter, a configuration of the detecting device 3 will be described using an XYZ coordinate system. The X-axis corresponds to an axis along a long side (one side) of the case 40 having a rectangular outer shape. The Y-axis is orthogonal to the X-axis and corresponds to an axis along the short side (the other side) of the case 40. The Z-axis is orthogonal to each of the X-axis and the Y-axis and corresponds to an axis along the normal line of the detection surface 16 that comes into contact with the measurement site M.

As illustrated in FIG. 3 and FIG. 4, the case 40 is a member that houses each constituent (the light-emitting unit section 11 and the light-receiving unit 12) constituting the detecting device 3. The case 40 has a box shape including a rectangular flat plate-shaped bottom surface portion 40a and a rectangular frame-shaped side plate portion 40b protruding from the periphery of the bottom surface portion 40a to the +Z side. The case 40 is formed of aluminum, for example. An inner circumferential surface 40b1 of the side plate portion 40b is colored in black and thus has light-shielding properties. As a result, reflection on the inner circumferential surface 40b1 of the side plate portion 40b is suppressed.

Note that the material and manufacturing method of the case 40 may be selected as desired. For example, the case 40 can be formed by injection molding of a resin material. Furthermore, a configuration in which the case 40 is formed integrally with the housing unit 1 is also suitable.

The light-emitting unit section 11 and the light-receiving unit 12 are installed on the bottom surface portion 40a of the case 40 in a state of being mounted on a wiring substrate (not illustrated). The light-shielding wall 41 is disposed between the light-emitting unit section 11 and the light-receiving unit 12 in the direction along the X-axis. The light-shielding wall 41 is a plate-shaped member protruding from the bottom surface portion 40a to the +Z side and extending in the Y-axis direction. The light-shielding wall 41 divides the housing space inside the case 40 into two in the X-axis direction. That is, the light-shielding wall 41 is a member that sets apart the space housing the light-emitting unit section 11 and the space housing the light-receiving unit 12 in the direction along the X-axis. The light-shielding wall 41 is a member having light-shielding properties for blocking light so as to prevent light emitted from the light-emitting unit section 11 from being directly incident on the light-receiving unit 12.

In the present embodiment, the light-shielding wall 41 is provided between the light-emitting unit section 11, which includes a first light-emitting unit 50 and a second light-emitting unit 60, and the light-receiving unit 12 in the direction along the X-axis. It can also be said that the light-shielding wall 41 is a member that shields a portion of green light LG, red light LR, and near-infrared light LI.

The sealing layer 42 is an optically transparent resin material filled into a gap between the light-emitting unit section 11 and the light-receiving unit 12, which are housed within the case 40, and the side plate portion 40b. The sealing layer 42 seals (molds) the light-emitting unit section 11 and the light-receiving unit 12 within the case 40. The surface of the sealing layer 42 functions as the detection surface 16.

Note that instead of a configuration in which the sealing layer 42 is used to seal, a configuration may be employed in which the upper surface of the side plate portion 40b of the case 40 is covered by a transmissive substrate. In this case, the upper surface of the transmissive substrate functions as the detection surface 16.

The light-emitting unit section 11 includes the first light-emitting unit 50, the second light-emitting unit 60, and a third light-emitting unit 70. The first light-emitting unit 50, the second light-emitting unit 60, and the third light-emitting unit 70 are light sources that each emit light of a different wavelength to the measurement site M.

The first light-emitting unit 50 ejects green light (first light) LG having a green wavelength band of 520 nm to 550 nm toward the measurement site M. The green light LG of the present embodiment is, for example, light having a peak wavelength of 520 nm.

The second light-emitting unit 60 ejects red light (second light) LR having a red wavelength band of 600 nm to 800 nm toward the measurement site M, for example. The red light LR of the present embodiment is, for example, light having a peak wavelength of 660 nm.

The third light-emitting unit 70 ejects near-infrared light (third light) LI having a near-infrared wavelength band of 800 nm to 1300 nm toward the measurement site M, for example. The near-infrared light LI of the present embodiment is, for example, light having a peak wavelength of 905 nm.

As the light-emitting elements constituting the first light-emitting unit 50, the second light-emitting unit 60, and the third light-emitting unit 70, a bare chip-type or a shell-type light-emitting diode (LED) is suitably utilized, for example. Note that the wavelength of light ejected by each light-emitting unit is not limited to the above-described numerical range. Hereinafter, when the first light-emitting unit 50, the second light-emitting unit 60, and the third light-emitting unit 70 are not particularly distinguished, they are collectively referred to as the light-emitting units 50, 60, and 70.

Each of the light-emitting units 50, 60, and 70 is installed in the case 40 so that the light-emitting surface thereof is parallel to the XY plane. That is, each of the light-emitting units 50, 60, and 70 emits light toward the +Z side.

Each of the light-emitting units 50, 60, and 70 emits light by the driving current supplied from the driving circuit 13 illustrated in FIG. 2. In the case of the present embodiment, the driving circuit 13 causes each of the light-emitting units 50, 60, and 70 to emit light independently in time sequence. Hereinafter, an aspect in which each of the light-emitting units 50, 60, and 70 independently emits light in time sequence is referred to by saying that the light-emitting units 50, 60, and 70 emits light in time sequence.

Light emitted from each of the light-emitting units 50, 60, and 70 is incident on the measurement site M. After being propagated through repeated reflections and scatterings inside the measurement site M, the light is emitted to the housing unit 1 side to reach the light-receiving unit 12. That is, the detecting device 3 according to the present embodiment is a reflection-type optical sensor in which the light-emitting unit section 11 and the light-receiving unit 12 are positioned on one side of the measurement site M.

As illustrated in FIG. 3, the light-emitting units 50, 60, and 70 are disposed side by side and spaced apart from each other in the direction along the Y-axis (first direction). Specifically, the second light-emitting unit 60 is disposed on the +Y side of the first light-emitting unit 50, while the third light-emitting unit 70 is disposed on the −Y side of the first light-emitting unit 50. That is, the first light-emitting unit 50 is disposed between the second light-emitting unit 60 and the third light-emitting unit 70 in the direction along the Y-axis. Furthermore, it can also be said that the first light-emitting unit 50 is positioned between the second light-emitting unit 60 and the third light-emitting unit 70.

By the way, traditionally, when acquiring both the pulse peak interval (PPI) and oxygen saturation (SpO2) as biological information of a test subject in a detecting device used in a measuring device, a light-receiving unit for green light (hereinafter referred to as the green light-receiving unit) for identifying a pulse wave, and a light-receiving unit for red light and near-infrared light (hereinafter referred to as the red and near-infrared light-receiving unit) for identifying oxygen saturation are respectively provided. This causes detecting devices to be upsized and makes it difficult to downsize measuring devices.

In recent years, there has been a demand for further downsizing measuring devices. In view of such background, the present inventors conducted diligent research on small detecting devices capable of acquiring both the pulse peak interval and oxygen saturation.

First, the present inventors focused on the fact that the transmittance of skin varies by the wavelength band of light.

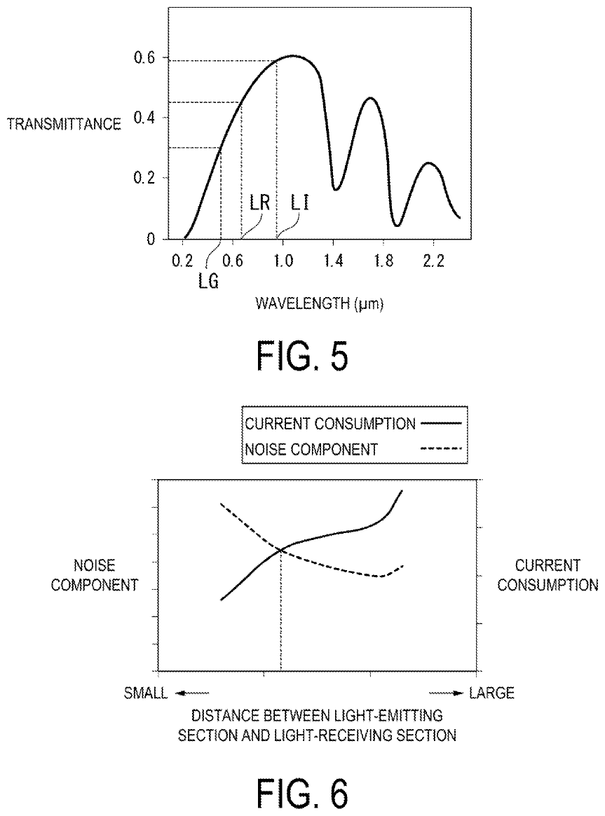

FIG. 5 is a graph showing a transmission spectrum of a skin. In FIG. 5, the horizontal axis indicates the wavelength of light, while the vertical axis indicates the transmittance (unit: %). FIG. 5 shows the transmission spectrum when the skin thickness is 0.43 mm as an example.

As illustrated in FIG. 5, the transmittance of the wavelength band of the green light LG (e.g., 520 nm) when incident on the skin is approximately 30%; the transmittance of the wavelength band of the red light LR (e.g., 660 nm) when incident on the skin is approximately 50% to 60%; and the transmittance of the wavelength band of the near-infrared light LI (e.g., 905 nm) when incident on the skin is approximately 60%.

The graph shown in FIG. 5 shows that the distance that light can propagate in a living body differs per wavelength of light. That is, according to the graph of FIG. 5, it can be seen that the green light LG can only propagate a short distance in the living body compared to the red light LR or the near-infrared light LI. In other words, it can also be said that the red light LR and the near-infrared light LI can propagate farther in the living body than the green light LG. Note that in FIG. 5, an example is given in which the skin thickness is 0.43 mm. However, even when the skin thickness is different, the red light LR and the near-infrared light LI can similarly propagate farther in the living body than the green light LG.

As shown in the graph of FIG. 5, the present inventors discovered that the green light LG is easily attenuated as it passes through the living body compared to the red light LR and the near-infrared light LI.

Furthermore, the present inventors simulated the state of incidence on the light-receiving unit of the green light that passed through the living body. Note that as a simulation condition, a traditionally-used regular-sized light-receiving unit was used.

As a result of this simulation, it was found that while the green light LG was favorably incident on a region on the light-emitting unit side of the light-receiving unit, the green light LG was not efficiently incident on a region on the side away from the light-emitting unit of the light-receiving unit. This is because green light was attenuated before being incident on the region away from the light-emitting unit.

The present inventors discovered that the green light LG that passed through the living body is easily attenuated, so the light-receiving region that receives the green light LG may be disposed in the vicinity of the light-emitting unit.

Furthermore, the present inventors focused on the fact that the noise component contained in the red light LR or the near-infrared light LI that propagates in the living body and that is incident on a light-receiving unit changes in accordance with the distance from the light-emitting unit to the light-receiving unit. In the following description, the red light LR is used as an example. However, the same applies to the near-infrared light LI.

FIG. 6 is a graph illustrating a relationship among the distance from a red light-emitting unit that emits red light to a light-receiving unit, the noise component of red light, and the current consumption of the red light-emitting unit. In FIG. 6, the horizontal axis indicates the distance from the red light-emitting unit to the light-receiving unit; the vertical axis on the left side indicates the noise component of the red light LR; and the vertical axis on the right side indicates the current consumption of the light-emitting unit.

As illustrated in FIG. 6, the closer the distance between the light-receiving unit and the light-emitting unit is, the more the noise component of the red light LR received at the light-receiving unit increases. In other words, the farther away the light-receiving unit is disposed from the light-emitting unit, the more the noise component of the red light LR decreases, which improves the detection accuracy for the red light LR. This is because on a light-receiving unit disposed close to the light-emitting unit, a red light component that is reflected at the surface layer portion of the living body and thus does not pass through the blood is incident. Such red light LR that does not pass through the blood is a noise component in identifying blood oxygen concentration at the light-receiving unit.

Accordingly, disposing the light-receiving unit away from the light-emitting unit can reduce the noise component contained in the red light LR and improve the detection accuracy for the red light LR. On the other hand, when the light-receiving unit is disposed away from the light-emitting unit, the distance to be propagated in the living body increases. Therefore, the input current to the light-emitting unit needs to be increased to increase the luminance of red light. In this case, since current consumption of the light-emitting unit increases, the distance between the light-emitting unit and the light-receiving unit may be determined taking into account the balance between the noise component and the current consumption. For example, in the case of the present embodiment, the distance between the light-receiving unit and the red light-emitting unit was set to a distance at which a curve indicating the noise component and a curve indicating the current consumption intersects.

Note that, for the near-infrared light LI as well, similar to the red light LR, disposing a near-infrared light-emitting unit that emits the near-infrared light LI and the light-receiving unit away from each other can improve the detection accuracy for the near-infrared light LI. Furthermore, the distance between the near-infrared light-emitting unit and the light-receiving unit may also be determined taking into account the balance between the noise component and the current consumption.

The present inventors discovered that disposing a light-receiving region that receives the red light LR or the near-infrared light LI as far as possible from the light-emitting unit can improve the detection accuracy of the light-receiving unit.

Based on the above-described discoveries, the present inventors completed the detecting device 3 and the measuring device 100 according to the present embodiment. For the detecting device 3 according to the present embodiment, a configuration is employed in which the light-receiving region of a traditionally-used regular-sized light-receiving unit 12 is divided into two, with one region close to the light-emitting unit side utilized as a light-receiving region for green light, and the other region away from the light-emitting unit side utilized as a light-receiving region for red and near-infrared light.

Hereinafter, a configuration of the light-receiving unit 12 of the present embodiment will be described.

The light-receiving unit 12 receives light caused by the light emission of the light-emitting unit section 11 and coming from the measurement site M. The light-receiving unit 12 of the present embodiment includes a first light-receiving region 51 and a second light-receiving region 61. The light-receiving unit 12 generates a detection signal in accordance with the intensity of the light received in the first light-receiving region 51 and the second light-receiving region 61, respectively. Hereinafter, when the light-receiving region 51 and the light-receiving region 61 are not particularly distinguished, they are collectively referred to as the “light-receiving regions 51 and 61”.

The light-receiving unit 12 is installed in the case 40 so that the light-receiving surface of each of the light-receiving regions 51 and 61 is parallel to the XY plane. That is, each of the light-receiving regions 51 and 61 is configured to receive light incident from the Z direction.

As illustrated in FIG. 3, the light-receiving regions 51 and 61 are disposed side by side and spaced apart from each other in the direction along the X-axis (second direction) that intersects (is orthogonal to) the Y-axis. Specifically, the first light-receiving region 51 is positioned on the +X side of the light-emitting unit section 11, while the second light-receiving region 61 is positioned on the +X side of the first light-receiving region 51. The second light-receiving region 61 is disposed on a side opposite to the light-emitting unit section 11 with the first light-receiving region 51 interposed therebetween. In the case of the present embodiment, the second light-receiving region 61 is provided at a position farther away from the first light-emitting unit 50 than the first light-receiving region 51. Specifically, the first light-receiving region 51 is positioned further toward the first light-emitting unit 50 side than the second light-receiving region 61 in the direction along the X-axis.

Here, the distance from the first light-emitting unit 50 to the first light-receiving region 51 is D1; the distance from the second light-emitting unit 60 to the second light-receiving region 61 is D2; and the distance from the third light-emitting unit 70 to the second light-receiving region 61 is D3. The distance D1 corresponds to the distance between the center portions of the first light-emitting unit 50 and the first light-receiving region 51 when viewed in plan view from the Z-axis direction. Furthermore, the distance D2 corresponds to the distance between the center portions of the second light-emitting unit 60 and the second light-receiving region 61 when viewed in plan view from the Z-axis direction. Furthermore, the distance D3 corresponds to the distance between the center portions of the third light-emitting unit 70 and the second light-receiving region 61 when viewed in plan view from the Z-axis direction.

In the detecting device 3 according to the present embodiment, the distance D1 from the first light-emitting unit 50 to the first light-receiving region 51 is shorter than the distance D2 from the second light-emitting unit 60 to the second light-receiving region 61. Furthermore, the distance D1 from the first light-emitting unit 50 to the first light-receiving region 51 is shorter than the distance D3 from the third light-emitting unit 70 to the second light-receiving region 61. Note that the distance D2 and the distance D3 are equal.

In this way, for the detecting device 3 according to the present embodiment, a configuration is employed in which the first light-receiving region 51 for receiving the green light LG is disposed at the position closest to the first light-emitting unit 50 that emits the green light LG.

In the case of the present embodiment, the first light-receiving region 51 and the second light-receiving region 61 are constituted by regions obtained by dividing the light incident region of the light-receiving unit 12 into two. The areas of the first light-receiving region 51 and the second light-receiving region 61 are equal to each other. The first light-receiving region 51 has such a plane area that an amount of light that is approximately 80% of the green light LG that passed through a living body can be received therein. According to the light-receiving unit 12 of the present embodiment, the green light LG that passed through the living body is incident on the first light-receiving region 51 at a sufficient amount of light. Thus, in the detecting device 3 according to the present embodiment, since there is no need to increase current consumption of the first light-emitting unit 50 in order to increase the light emission amount of the green light LG, power consumption of the light-emitting unit section 11 can be reduced.

As illustrated in FIG. 4, the light-receiving unit 12 includes a light-receiving element 120, an angle-limiting filter 121, and a band-pass filter (first filter) 122.

The light-receiving element 120 is constituted by, for example, a photodiode (PD). The angle-limiting filter 121 is provided so as to cover the entire light-receiving surface 120a of the light-receiving element 120. The angle-limiting filter 121 is formed, for example, by embedding a plug 1212 formed of a material having light-shielding properties such as tungsten in a silicon oxide layer 1211 having optical transparency.

The silicon oxide layer 1211 forms an optical path that guides light to the light-receiving surface 120a of the light-receiving element 120. The plug 1212 embedded in the silicon oxide layer 1211 limits the incident angle of light passing through the optical path (the silicon oxide layer 1211). That is, when the light incident into the silicon oxide layer 1211 is tilted relative to the optical path by a predetermined angle or more, the incident light impinges on the plug 1212, causing a portion of that light to be absorbed by the plug 1212 and the remainder to be reflected. Further, since the intensity of the reflected light grows weak through repeated reflections before passing through the optical path, light that can ultimately pass through the angle-limiting filter 121 is substantially limited to light that is tilted relative to the light path by a predetermined limit angle or less.

The angle-limiting filter 121 has a characteristic of transmitting light incident at an angle smaller than a predetermined incident angle, and cutting, rather than transmitting, light incident at an angle greater than a predetermined incident angle. As a result, the angle-limiting filter 121 is capable of limiting the incident angle of light incident on the light-receiving element 120. Specifically, the angle-limiting filter 121 transmits light that propagates in the living body and thus is incident at a predetermined incident angle (hereinafter referred to as the permissible incident angle), and cuts light that is incident at an angle greater than the permissible incident angle, including outside light such as sunlight and light that was not incident on the living body.

The band-pass filter 122 is provided in a region corresponding to the first light-receiving region 51 of the light-receiving surface 120a of the light-receiving element 120. The band-pass filter 122 has a characteristic of selectively transmitting a wavelength band of the green light LG, and absorbing and thereby cutting the red light LR and the near-infrared light LI, which fall under light in the other wavelength bands. The band-pass filter 122 is formed, for example, by alternately stacking a plurality of low refractive index layers such as silicon oxide and a plurality of high refractive index layers such as titanium oxide on the angle-limiting filter 121. Note that the band-pass filter 122 is formed at a region corresponding to the first light-receiving region 51 using a traditionally known photolithographic process.

On the other hand, in the light-receiving unit 12, the second light-receiving region 61 is not provided with a band-pass filter that selectively transmits the red light LR or the near-infrared light LI, but is provided with only the angle-limiting filter 121. Thus, the light-receiving unit 12 can limit the incident angle of the red light LR or the near-infrared light LI reaching the light-receiving element 120 in the second light-receiving region 61. For example, the angle-limiting filter 121 transmits the red light LR or the near-infrared light LI that propagates in the living body and is incident at the permissible incident angle, and cuts light that is incident at an angle greater than the permissible incident angle, including outside light such as sunlight and the red light LR or the near-infrared light LI that did not pass through the living body.

As illustrated in FIG. 2, in the case of the present embodiment, the light-receiving unit 12 receives light of each of the light-emitting units 50, 60, and 70 that are driven in time division in synchronization with the light emission timing thereof, and generates a detection signal in accordance with each light.

The light-receiving unit 12 sends the detection signal generated at each of the light-receiving units 51 and 61 to the output circuit 14. The output circuit 14 includes, for example, an A/D converter that converts the detection signal generated by each of the light-receiving regions 51 and 61 from analog to digital, and an amplification circuit that amplifies the converted detection signal (both not illustrated). The output circuit 14 generates a plurality of detection signals S (S1, S2, and S3) corresponding to different wavelengths.

Here, the detection signal S1 is a signal that represents the received light intensity of the first light-receiving region 51 when the green light LG emitted from the first light-emitting unit 50 is received. The detection signal S2 is a signal that represents the received light intensity of the second light-receiving region 61 when the red light LR emitted from the second light-emitting unit 60 is received. The detection signal S3 is a signal that represents the received light intensity of the second light-receiving region 61 when the near-infrared light LI emitted from the third light-emitting unit 70 is received.

In general, since the absorbance of blood varies between the diastolic phase and the systolic phase of the blood vessel, each of the detection signals S is a pulse wave signal that includes a periodic fluctuation component corresponding to the pulsating component (plethysmogram) of an artery inside the measurement site M.

Note that the driving circuit 13 and the output circuit 14 are mounted on the wiring substrate together with the light-emitting unit section 11 and the light-receiving unit 12 in the form of an integrated circuit (IC) chip. Note that, as described above, the driving circuit 13 and the output circuit 14 may be installed outside of the detecting device 3.

The control device 5 is an arithmetic processing device such as a central processing unit (CPU) and a field-programmable gate array (FPGA). The control device 5 controls the entire measuring device 100. The storage device 6 is constituted, for example, by a non-volatile semiconductor memory. The storage device 6 stores a program executed by the control device 5 and various data used by the control device 5. Note that a configuration in which the functions of the control device 5 are distributed among a plurality of integrated circuits or a configuration in which some or all of the functions of the control device 5 are realized by a dedicated electronic circuit can also be employed. Note that in FIG. 2, the control device 5 and the storage device 6 are illustrated as separate constituents. However, it is also possible to realize the control device 5 including the storage device 6 by, for example, an application-specific integrated circuit (ASIC) and the like.

The control device 5 of the present embodiment executes a program stored in the storage device 6, and thereby identifies biological information of the test subject from the plurality of detection signals S (S1, S2, and S3) generated by the detecting device 3.

Specifically, the control device (information analysis unit) 5 identifies a pulse wave of the test subject from the detection signal S1 that represents the received light intensity of the green light LG received by the first light-receiving region 51. The controller 5 can identify a pulse peak interval (PPI) of the test subject based on, for example, the detection signal S1. Furthermore, the control device 5 can analyze the detection signal S2 that represents the received light intensity of the red light LR received by the second light-receiving region 61, and the detection signal S3 that represents the received light intensity of the near-infrared light LI received by the second light-receiving region 61, and thereby identify oxygen saturation (SpO2) of the test subject.

As described above, in the measuring device 100, the control device 5 functions as an information analysis unit that identifies biological information from detection signals S indicating detection results by the detecting device 3. The control device 5 causes the display device 4 to display biological information identified from detection signals S. Note that it is also possible to notify a user of a measurement result by sound output. A configuration in which a user is notified of a warning (possibility of impairment in a bodily function) when the pulse rate or oxygen saturation fluctuates to a numerical value outside a predetermined range is also suitable.

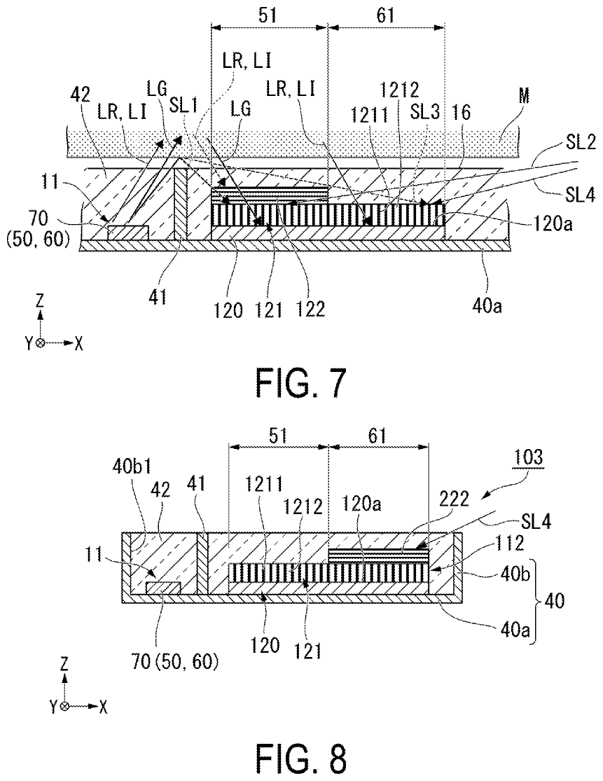

FIG. 7 is a view for explaining an operation of the detecting device 3.

As illustrated in FIG. 7, in the detecting device 3 according to the present embodiment, a portion of the green light LG emitted from the first light-emitting unit 50 is reflected by the surface of the living body (measurement site M) and thus is sometimes directly incident on the first light-receiving region 51 without passing through the living body. Furthermore, outside light such as sunlight is sometimes directly incident on the first light-receiving region 51 by passing through the gap between the living body and the detection surface 16. Hereinafter, the green light LG heading to the first light-receiving region 51 without passing through the living body is referred to as the “first stray light component SL1”. Outside light directly heading to the first light-receiving region 51 is referred to as the “second stray light component SL2”.

Since it has a green wavelength band, the first stray light component SL1 passes through the band-pass filter 122 and is incident on the angle-limiting filter 121 provided at a lower layer of the band-pass filter 122. As described above, the angle-limiting filter 121 has a characteristic of transmitting light incident at an angle smaller than the permissible incident angle, and cutting light incident at an angle greater than the permissible incident angle.

Since the first stray light component SL1 is incident on the first light-receiving region 51 without passing through the living body, the incident angle of the green light LG relative to the first light-receiving region 51 is greater than the permissible incident angle of the angle-limiting filter 121. In other words, the first stray light component SL1 is cut by the angle-limiting filter 121. As a result, the first light-receiving region 51 can suppress the first stray light component SL1 from being incident on the light-receiving surface 120a of the light-receiving element 120 by the angle-limiting filter 121.

The second stray light component SL2 is generally cut by the band-pass filter 122. However, the component having a green wavelength band contained in the second stray light component SL2 is transmitted through the band-pass filter 122. Here, as described above, since the second stray light component SL2 is incident by passing through the gap between the living body and the detection surface 16, the incident angle of the second stray light component SL2 relative to the first light-receiving region 51 is greater than the permissible incident angle of the angle-limiting filter 121. Therefore, a portion of the second stray light component SL2 transmitted through the band-pass filter 122 (the component having a green wavelength band) is cut by the angle-limiting filter 121. As a result, the first light-receiving region 51 can suppress the second stray light component SL2 from being incident on the light-receiving surface 120a of the light-receiving element 120 by the angle-limiting filter 121.

In this way, in the detecting device 3 according to the present embodiment, it is possible to cause the green light LG ejected from the light-emitting unit section 11 and passed through the living body to be efficiently incident on the light-receiving surface 120a of the light-receiving element 120. Furthermore, in the detecting device 3 according to the present embodiment, it can be made difficult for the first stray light component SL1 and the second stray light component SL2 to be incident on the light-receiving surface 120a of the light-receiving element 120.

Thus, with the first stray light component SL1 and the second stray light component SL2 that represent noise components suppressed from being incident, the first light-receiving region 51 can obtain a high signal/noise (S/N) ratio. Therefore, in the detecting device 3 according to the present embodiment, since the green light LG can be received with high accuracy in the first light-receiving region 51, the light emission amount of the green light LG at the first light-emitting unit 50 can be restrained to suppress power consumption of the light-emitting unit section 11.

Furthermore, a portion of the red light LR emitted from the second light-emitting unit 60 or a portion of the near-infrared light LI emitted from the third light-emitting unit 70 is sometimes directly incident on the second light-receiving region 61 without passing through the living body. Furthermore, outside light such as sunlight is sometimes directly incident on the second light-receiving region 61 by passing through the gap between the living body and the detection surface 16. Hereinafter, the red light LR or the near-infrared light LI heading directly to the second light-receiving region 61 without passing through the living body is collectively referred to as the “third stray light component SL3”. Outside light heading directly to the second light-receiving region 61 is referred to as the “fourth stray light component SL4”.

Since the third stray light component SL3 is incident on the angle-limiting filter 121 without passing through the living body, the incident angle of the third stray light component SL3 relative to the second light-receiving region 61 is greater than the permissible incident angle of the angle-limiting filter 121. Furthermore, since the fourth stray light component SL4 is incident by passing through the gap between the living body and the detection surface 16, the incident angle of the fourth stray light component SL4 relative to the second light-receiving region 61 is greater than the permissible incident angle of the angle-limiting filter 121.

Therefore, the third stray light component SL3 and the fourth stray light component SL4 are successfully cut by the angle-limiting filter 121. As a result, the second light-receiving region 61 can suppress the third stray light component SL3 and the fourth stray light component SL4 from being incident on the light-receiving surface 120a of the light-receiving element 120 by the angle-limiting filter 121.

In this way, in the detecting device 3 according to the present embodiment, it is possible to cause the red light LR or the near-infrared light LI emitted from the light-emitting unit section 11 and passed through the living body to be efficiently incident on the light-receiving surface 120a of the light-receiving element 120. Furthermore, in the detecting device 3 according to the present embodiment, it can be made difficult for the third stray light component SL3 and the fourth stray light component SL4 to be incident on the light-receiving surface 120a of the light-receiving element 120.

Thus, with the third stray light component SL3 and the fourth stray light component SL4 that represent noise components suppressed from being incident, the second light-receiving region 61 can obtain a high S/N ratio. According to the detecting device 3 according to the present embodiment, since the red light LR and the near-infrared light LI are efficiently received in the second light-receiving region 61, the light emission amount of each of the second light-emitting unit 60 and the third light-emitting unit 70 can be restrained to suppress power consumption of the light-emitting unit section 11.

Furthermore, in the detecting device 3 according to the present embodiment, the distance between the second light-emitting unit 60 and the second light-receiving region 61 and the distance between the third light-emitting unit 70 and the second light-receiving region 61 (distance D2 and distance D3) are greater than the distance D1 between the first light-emitting unit 50 and the first light-receiving region 51. In other words, the distances that the red light LR and the near-infrared light LI propagate in the living body before being incident on the second light-receiving region 61 are greater than the distance that the green light LG propagates in the living body before being incident on the first light-receiving region 51.

As shown in FIG. 4, with the red light LR or the near-infrared light LI, the longer the propagation distance in the living body is, the more the component that is reflected at the surface layer portion of the living body and thus does not pass through the blood, that is, a noise component in identifying blood oxygen concentration decreases. Therefore, with the noise component suppressed from being incident, the second light-receiving region 61 can obtain a high S/N ratio. Thus, in the detecting device 3 according to the present embodiment, the red light LR or the near-infrared light LI can be received with high accuracy in the second light-receiving region 61.

On the other hand, when the propagation distance in the living body of the red light LR or the near-infrared light LI is too long, it becomes necessary to increase the light emission amount of the second light-emitting unit 60 or the third light-emitting unit 70. In the case of the present embodiment, by forming the second light-receiving region 61 together with the first light-receiving region 51 on the light-receiving surface 120a of one light-receiving element 120, the second light-receiving region 61 is disposed as close as possible to the second light-emitting unit 60 and the third light-emitting unit 70. As a result, while maintaining the light receiving accuracy for the red light LR and the near-infrared light LI, power consumption of the second light-emitting unit 60 or the third light-emitting unit 70 can be restrained to suppress power consumption of the light-emitting unit section 11.

Furthermore, in the case of the present embodiment, only the red light LR and the near-infrared light LI are incident on the second light-receiving region 61. Therefore, no band-pass filter that selectively transmits the red light LR and the near-infrared light LI and cuts the green light LG is provided in the second light-receiving region 61. That is, for the detecting device 3 according to the present embodiment, a configuration may be employed in which only the first light-receiving region 51 includes the band-pass filter 122 and the second light-receiving region 61 includes no band-pass filter. Thus, in the detecting device 3 according to the present embodiment, the band-pass filter for the second light-receiving region 61 can be omitted to reduce cost.

As described above, according to the detecting device 3 according to the present embodiment, even when the light emission amounts of the light-emitting units 50, 60, and 70 are restrained to reduce power consumption, light passed through the living body can be received with high accuracy at the light-receiving unit 12. Furthermore, in the detecting device 3 according to the present embodiment, the band-pass filter in the second light-receiving region 61 can be omitted to reduce cost.

When the green light LG emitted from the first light-emitting unit 50 and propagated inside the measurement site M is received in the first light-receiving region 51, the light-receiving unit 12 generates a detection signal in accordance with the received light intensity. Note that while a portion of the green light LG is incident on the second light-receiving region 61, the green light LG incident on the second light-receiving region 61 is cut by the angle-limiting filter 121.

Furthermore, when the red light LR emitted from the second light-emitting unit 60 and propagated inside the measurement site M or the near-infrared light LI emitted from the third light-emitting unit 70 and propagated inside the measurement site M is received in the second light-receiving region 61, the light-receiving unit 12 generates a detection signal in accordance with the received light intensity. Note that while a portion of the red light LR and the near-infrared light LI is incident on the first light-receiving region 51, the red light LR and the near-infrared light LI incident on the first light-receiving region 51 are cut by the band-pass filter 122.

As described above, the detecting device 3 according to the present embodiment includes: the first light-emitting unit 50 configured to emit the green light LG; the second light-emitting unit 60 configured to emit the red light LR having a wavelength band higher than that of the green light LG; and the light-receiving unit 12 configured to receive the green light LG emitted from the first light-emitting unit 50 and emitted from the measurement site M and the red light LR emitted from the second light-emitting unit 60 and emitted from the measurement site M. The light-receiving unit 12 includes the first light-receiving region 51 configured to receive the green light LG, the second light-receiving region 61 provided at a position farther away from the first light-emitting unit 50 than the first light-receiving unit 51 and configured to receive the red light LR, and the band-pass filter 122 provided in the first light-receiving region 51 and configured to selectively transmit the green light LG.

In the case of the present embodiment, the band-pass filter 122 is a band-pass filter that selectively transmits the green light LG. Furthermore, the detecting device 3 according to the present embodiment further includes the third light-emitting unit 70 configured to emit the near-infrared light LI, wherein the first light-emitting unit 50, the second light-emitting unit 60, and the third light-emitting unit 70 each independently emits light in time sequence.

According to the detecting device 3 according to the present embodiment, the green light LG, the red light LR, and the near-infrared light LI can be each received by utilizing one light-receiving unit 12. Thus, compared to a configuration in which two light-receiving units are used as in the related art, the device configuration can be downsized to approximately half, for example. Thus, a small detecting device 3 capable of acquiring both the pulse peak interval and oxygen saturation can be provided. Furthermore, confining the number of light-receiving unit 12 to one can reduce the cost of the detecting device 3.

In the present embodiment, the light-receiving unit 12 receives each of the green light LG, the red light LR, and the near-infrared light LI in synchronization with the light emission timing thereof.

According to this configuration, signals of the green light LG, the red light LR, and the near-infrared light LI can be acquired in a temporally separated manner. Thus, each light is suppressed from acting as noise for any of the other light. As a result, a configuration can be realized in which both the pulse peak interval and oxygen saturation can be detected using one light-receiving unit 12 while downsizing the device configuration.

### Second Embodiment

Next, a second embodiment will be described. In the first embodiment, an example is given of a case in which the band-pass filter 122 is provided in the first light-receiving region 51. However, the detecting device according to the present embodiment differs from that of the first embodiment in that the first filter is provided only in the second light-receiving region 61.

FIG. 8 is a cross-sectional view of a detecting device according to the present embodiment. FIG. 8 is a configuration corresponding to FIG. 4 in the first embodiment. Note that configurations and members common to the first embodiment will be given an identical reference numeral and detailed description thereof will be omitted.

As illustrated in FIG. 8, the detecting device 103 according to the present embodiment includes a band-pass filter (first filter) 222 provided in the second light-receiving region 61 of the light-receiving unit 112. The band-pass filter 222 is provided in a region corresponding to the second light-receiving region 61 of the light-receiving surface 120a of the light-receiving element 120. The band-pass filter 222 has a characteristic of selectively transmitting the red light LR and the near-infrared light LI, and absorbing and thereby cutting light in the other wavelength bands. The band-pass filter 222 is formed, for example, by alternately stacking a plurality of low refractive index layers such as silicon oxide and a plurality of high refractive index layers such as titanium oxide on the angle-limiting filter 121. Note that the band-pass filter 222 is formed at a region corresponding to the second light-receiving region 61 using a traditionally known photolithographic process.

The second light-receiving region 61 of the light-receiving unit 112 is disposed closer to the side plate portion 40b of the case 40 than the first light-receiving region 51. Therefore, the fourth stray light component SL4 is easily incident on the second light-receiving region 61 by passing through the gap between the living body and the detection surface 16.

According to the detecting device 103 according to the present embodiment, the band-pass filter 222 including a band-pass filter provided in the second light-receiving region 61 can make it difficult for the fourth stray light component SL4 incident on the second light-receiving region 61 to reach the light-receiving surface 120a.

For example, when the distance between the side plate portion 40b and the second light-receiving region 61 is decreased and thereby the case 40 is decreased in size, the effect of the fourth stray light component SL4 is facilitated. In the case of the detecting device 103 according to the present embodiment, the effect of the fourth stray light component SL4 is restrained by the band-pass filter 222 provided in the second light-receiving region 61. Thus, in the detecting device 103 according to the present embodiment, the case 40 can be decreased in size and thereby the device configuration can be downsized while suppressing the effect of the noise component due to the fourth stray light component SL4.

### Third Embodiment

Next, a third embodiment will be described. In the first embodiment, an example is given of a case in which the band-pass filter 122 is provided in the first light-receiving region 51. However, the detecting device according to the present embodiment differs from that of the first embodiment in that a band-pass filter is provided in each of the first light-receiving region 51 and the second light-receiving region 61.

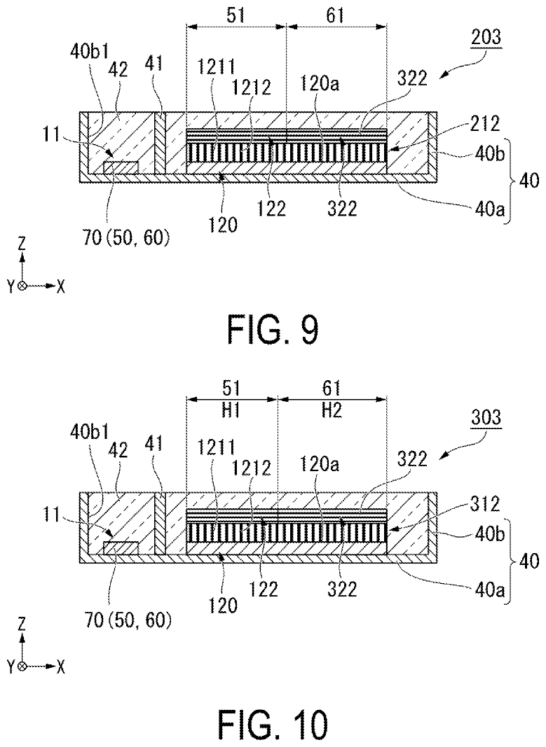

FIG. 9 is a cross-sectional view of a detecting device according to the present embodiment. FIG. 9 is a configuration corresponding to FIG. 4 in the first embodiment. Note that configurations and members common to the first embodiment will be given an identical reference numeral and detailed description thereof will be omitted.

As illustrated in FIG. 9, the detecting device 203 according to the present embodiment includes the band-pass filter (first filter) 122 provided in the first light-receiving region 51 of the light-receiving unit 212, and a band-pass filter (second filter) 322 provided in the second light-receiving region 61 of the light-receiving unit 212. The band-pass filter 122 has a characteristic of selectively transmitting a wavelength band of the green light LG, and absorbing and thereby cutting light in the other wavelength bands. The band-pass filter 322 has a characteristic of selectively transmitting the red light LR and the near-infrared light LI, and absorbing and thereby cutting light in the other wavelength bands.

The band-pass filter 122 and the band-pass filter 322 are formed at regions corresponding to the first light-receiving region 51 and the second light-receiving region 61, respectively, of the light-receiving surface 120a of the light-receiving element 120 by using a traditionally known photolithographic process.

According to the detecting device 203 according to the present embodiment, the first light-receiving region 51 and the second light-receiving region 61 are provided with the band-pass filters 122 and 322, respectively. Therefore, the effect of the noise component in the light-receiving regions 51 and 61 can be reduced. Thus, a detecting device capable of detecting both the pulse peak interval and oxygen saturation with high accuracy can be provided while downsizing the device configuration.

FIG. 10 is a cross-sectional view of a detecting device according to a modified example of the present embodiment.

As illustrated in FIG. 10, in a detecting device 303 according to the present modified example, a width H2 in the direction along the X-axis of the band-pass filter 322 provided in the second light-receiving region 61 is greater than a width H1 in the direction along the X-axis of the band-pass filter 122 provided in the first light-receiving region 51. In other words, in the case of the present modified example, unlike the above-described embodiments, the width of the second light-receiving region 61 is greater than the width of the first light-receiving region 51 in the direction along the X-axis. Note that the light emission amount of each of the light-emitting units 50, 60, and 70 may be adjusted in accordance with the widths of the second light-receiving region 61 and the first light-receiving region 51.

As described above, since the green light LG is easily attenuated as the distance to be propagated in the living body extends, the more a region is located to the +X side (the second light-receiving region 61 side) of the first light-receiving region 51, the less the incident amount of the green light LG is. In the case of the present modified example, with the +X side of the first light-receiving region 51 utilized as the second light-receiving region 61, the degree of increase in the amount of received red light LR and near-infrared light LI due to expansion of the second light-receiving region is greater than the degree of decrease in the amount of received green light LG. Therefore, according to the configuration of the present modified example, the detection accuracy for the red light LR and the near-infrared light LI can be relatively improved without changing the size of the light-receiving unit 12.

Thus far, the present disclosure has been described based on the embodiments described above. However, the present disclosure is not limited to the above-described embodiments, and can be carried out in various aspects without departing from the spirit and scope of the present disclosure.

For example, in the above-described embodiments, a human is used as an example of a living body. However, the present disclosure is also applicable to measurement of biological information (e.g., pulse) of other animals.

Furthermore, for the measuring device 100 according to the above-described embodiments, an example is given of a case in which the detecting device 3 is provided in the housing unit 1. However, the installation location of the detecting device 3 is not limited to this. For example, the detecting device 3 may be embedded in the belt 2.

Furthermore, for the measuring device 100 according to the above-described embodiments, a wristwatch-type configuration is given as an example. However, the present disclosure is also applicable, for example, to a configuration in which a necklace-type measuring device 100 is mounted around the neck of the test subject, a configuration in which a seal-type measuring device 100 is attached to the body of the test subject, or a configuration in which a head-mounted display-type measuring device 100 is mounted on the head of the test subject.

Furthermore, in the above-described embodiments, an example is given of a case in which the angle-limiting filter 121 is used in common for the first light-receiving region 51 and the second light-receiving region 61. However, an angle-limiting filter may be provided separately in the first light-receiving region 51 and the second light-receiving region 61. In this case, the permissible incident angles for the first light-receiving region 51 and the second light-receiving region 61 may be varied.

A detecting device according to one aspect of the present disclosure may have the following configuration.

A detecting device according to an aspect of the present disclosure includes: a first light-emitting unit configured to emit first light having a green wavelength band; a second light-emitting unit configured to emit second light having a wavelength band higher than that of the green wavelength band; and a light-receiving unit configured to receive the first light emitted from the first light-emitting unit and emitted from a living body and the second light emitted from the second light-emitting unit and emitted from the living body; wherein the light-receiving unit includes a first light-receiving region configured to receive the first light, a second light-receiving region provided at a position farther away from the first light-emitting unit than the first light-receiving region and configured to receive the second light, and a first filter provided in one of the first light-receiving region and the second light-receiving region and configured to selectively transmit light in a corresponding wavelength band.

The detecting device according to one aspect of the present disclosure may have a configuration in which a first filter is provided at the first light-receiving unit, and the first filter is a band-pass filter configured to selectively transmit the first light.

The detecting device according to one aspect of the present disclosure may have a configuration in which a first filter is provided in the second light-receiving unit, and the first filter is a band-pass filter configured to selectively transmit the second light.

The detecting device according to one aspect of the present disclosure may have a configuration in which the detecting device further includes: a third light-emitting unit configured to emit third light; wherein the second light-emitting unit emits light in a wavelength band of one of a red wavelength band and a near-infrared wavelength band as the second light, the third light-emitting unit emits light in a wavelength band of the other one of the red wavelength band and the near-infrared wavelength band as the third light, and the first light-emitting unit, the second light-emitting unit, and the third light-emitting unit each independently emits light in time sequence.

The detecting device according to one aspect of the present disclosure may have a configuration in which the light-receiving unit is configured to receive each of the first light, the second light, and the third light in synchronization with a light emission timing thereof.

The detecting device according to one aspect of the present disclosure may have a configuration in which the first light-emitting unit and the second light-emitting unit are disposed side by side in a first direction, the first light-receiving region and the second light-receiving region are disposed side by side in a second direction intersecting the first direction, and the first light-receiving region is positioned further toward the first light-emitting unit side than the second light-receiving region in the second direction.

The detecting device according to one aspect of the present disclosure may have a configuration in which the light-receiving unit further includes a second filter provided in the other one of the first light-receiving region and the second light-receiving region and configured to selectively transmit light in a corresponding wavelength band.

The detecting device according to one aspect of the present disclosure may have a configuration in which, a width in the second direction of one filter, of the first filter and the second filter, is greater than a width in the second direction of the other filter, and the one filter is provided in the second light-receiving region and the other filter is provided in the first light-receiving region.

A measuring device according to one aspect of the present disclosure may have the following configuration.

A measuring device according to one aspect of the present disclosure includes a detecting device according to any one of the above-described aspects; and an information analysis unit configured to identify biological information from a detection signal indicating a detection result by the detecting device.

What is claimed is:

1. A detecting device comprising:
a first light-emitting unit configured to emit first light having a green wavelength band;
a second light-emitting unit configured to emit second light having a wavelength band higher than that of the green wavelength band; and
a light-receiving unit configured to receive each of the first light emitted from the first light-emitting unit and emitted from a living body and the second light emitted from the second light-emitting unit and emitted from the living body; wherein
the light-receiving unit includes
a first light-receiving region configured to receive the first light,
a second light-receiving region provided at a position farther away from the first light-emitting unit than the first light-receiving region and configured to receive the second light, and
a first filter provided in one of the first light-receiving region and the second light-receiving region and configured to selectively transmit light in a corresponding wavelength band.

2. The detecting device according to claim 1, wherein
the first filter is provided in the first light-receiving region, and
the first filter is a band-pass filter configured to selectively transmit the first light.

3. The detecting device according to claim 1, wherein
the first filter is provided in the second light-receiving region, and
the first filter is a band-pass filter configured to selectively transmit the second light.

4. The detecting device according to claim 1, further comprising:
a third light-emitting unit configured to emit third light, wherein
the second light-emitting unit emits light in a wavelength band of one of a red wavelength band and a near-infrared wavelength band as the second light,
the third light-emitting unit emits light in a wavelength band of the other one of the red wavelength band and the near-infrared wavelength band as the third light, and
the first light-emitting unit, the second light-emitting unit, and the third light-emitting unit each independently emits light in time sequence.

5. The detecting device according to claim 4, wherein the light-receiving unit receives each of the first light, the second light, and the third light in synchronization with a light emission timing thereof.

6. The detecting device according to claim 1, wherein
the first light-emitting unit and the second light-emitting unit are disposed side by side in a first direction,
the first light-receiving region and the second light-receiving region are disposed side by side in a second direction intersecting the first direction, and
the first light-receiving region is positioned further toward the first light-emitting unit side than the second light-receiving region in the second direction.

7. The detecting device according to claim 6, wherein
the light-receiving unit further includes a second filter provided in the other one of the first light-receiving region and the second light-receiving region and
the second filter is a band-pass filter configured to selectively transmit light in a corresponding wavelength band.

8. The detecting device according to claim 7, wherein
a width in the second direction of one filter, of the first filter and the second filter, is greater than a width in the second direction of the other filter, the one filter being provided in the second light-receiving region, and the other filter being provided in the first light-receiving region.

9. A measuring device, comprising:
a detecting device according to claim 1; and
an information analysis unit configured to identify biological information from a detection signal indicating a detection result by the detecting device.
